(19) United States
(12) Patent Application Publication
(10) Pub. No.: US 2024/0082065 A1
(43) Pub. Date: Mar. 14, 2024

(54) WATERPROOF AND TRANSPARENT WOUND BARRIER AND METHOD OF MANUFACTURING SAME

(71) Applicant: Moteah Williams, Gainesville, FL (US)

(72) Inventor: Moteah Williams, Gainesville, FL (US)

(21) Appl. No.: 15/623,376

(22) Filed: Jun. 14, 2017

Related U.S. Application Data
(60) Provisional application No. 62/360,178, filed on Jul. 8, 2016.

(51) Int. Cl. A61F 13/02

(52) U.S. Cl. CPC A61F 13/0233; A61F 13/025; A61F 13/0263; A61F 2013/00868

(57) ABSTRACT

A wound barrier provides an adhesive sealed barrier surrounding a wound site that is oxygen permeable but which is substantially moisture vapor impermeable. The barrier includes a compound material that is a thin and compliant oxygen permeable backing material with pressure sensitive adhesive deposited on one surface. An adhesive deadening layer is island deposited onto the adhesive to form a non-adhesive zone and an adhesive zone at the periphery of the wound barrier. A liner layer is then placed over the adhesive side of the wound barrier to cover the adhesive until the barrier is to be applied to cover and seal off a wound site. The compound material can include a casting sheet to provide support while the barrier is applied and then removed to improve the compliant nature of the barrier.

### CROSS-REFERENCE TO RELATED APPLICATION

This application claims priority to U.S. Provisional Application Ser. No. 62/350,178 filed Jun. 14, 2016 and entitled “IMPROVED WATERPROOF TRANSPARENT BARRIER AND METHOD OF MANUFACTURING SAME,” and which is incorporated herein in its entirety by this reference.

### FIELD OF THE INVENTION

The invention relates to wound and incision cite protection, and more particularly to waterproof barriers for protecting such sites while a patient bathes or showers.

### BACKGROUND OF THE INVENTION

Patients often require long-term care of wounds such as stomas and ulcers, as well as surgical incisions, including PICC (percutaneous indwelling central catheter) lines, ports and catheters or other central venous insertion sites where the medical devices remain inserted for extended periods of time. Patients must eventually bathe or shower, but such sites must be protected from water to prevent infection.

Creating effective protection for such sites during exposure to water is made difficult because the devices must be highly resistant to water, yet they should also be permeable to oxygen to promote healing. The devices should be easy for caregivers to properly and effectively apply to patients without disturbing the wound, but should also be large enough and sufficiently compliant so that they effectively accommodate installed devices that are part of the wound site as well as permit a proper sea notwithstanding that the varying physiology of patients at the wound site.

### SUMMARY OF THE INVENTION

The wound barrier of the invention provides a waterproof wound barrier for isolating various types of wound sites from moisture while remaining oxygen permeable. The wound barrier of the invention is also sufficiently compliant to isolate wounds that include those associated with long-term installation of devices such as ports, PICC lines and catheters, by surrounding the entire site including the installed devices and any applied dressings, yet is easy for healthcare professionals to position and apply effectively. A method of manufacturing the invention ensures a strong and consistent adhesive seal around the wound site, ensures high resistivity to moisture penetration, while permitting transmission of oxygen, and facilitates ease of application by providing a temporary structural support that can be removed once applied.

The barrier includes a compound layer made up of a thin and compliant backing layer with an adhesive, such as medical grade pressure sensitive adhesive deposited on one surface of the backing material. The opposite surface is a casting sheet that provides additional structural integrity to the compound layer and the wound barrier overall to make it easier for healthcare professionals to handle and apply the barrier. Once positioned and applied, the casting sheet can be removed, thereby making the barrier more compliant while applied to a patient.

An adhesive deadening layer is island deposited to the adhesive surface to establish an adhesive free zone and an adhesive zone around the periphery of the barrier. The deadening layer can be a backing layer having a very low moisture vapor transmission rate, preferably below 10 grams per square meter per 24 hours. A release liner is then applied to the adhesive or applied side of the barrier to cover the adhesive until the barrier is to be applied.

In an embodiment, a tab can be score into the compound layer down to the casting sheet along one side of the barrier to facilitate removal of the casting sheet once the barrier is applied to isolate the wound site. In another embodiment, the casting sheet can be applied in an overlapping manner to facilitate application of the barrier to the patient.

### BRIEF DESCRIPTION OF THE DRAWINGS

FIG. 1 is an illustration of an embodiment of the wound barrier of the invention installed over the wound site of a patient;

FIG. 2A is a plan view of one embodiment of the wound barrier of the invention;

FIG. 2B is a cross-sectional view of the wound barrier of FIG. 2A, illustrating the layers of material that form the wound barrier of the invention;

FIG. 3A is a plan view of an alternative embodiment of the wound barrier of the invention that includes a pull tab by which to more easily remove a casting layer from the top of the wound barrier after installation;

FIG. 3B is a cross-sectional view of the wound barrier of FIG. 3A, illustrating the layers of material that form the wound barrier of the invention;

FIG. 4 is a procedural flow diagram illustrating a method of manufacturing the wound barrier of the invention.

### DETAILED DESCRIPTION

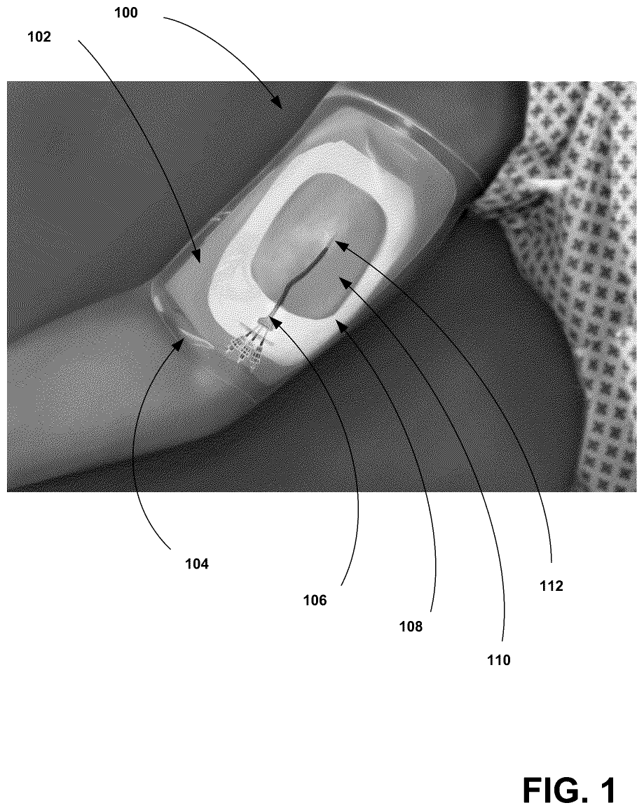

An embodiment of the wound barrier of the invention 100 is illustrated in FIG. 1 as installed over wound 112 of a patient that was created by insertion of a catheter 106. The wound barrier 100 forms a moisture barrier around the entire wound site that includes wound 112, the catheter device 106 and a wound dressing that includes frame 108 and wound cover 110. The wound dressing is intended to provide some protection from the environment, but is not an effective barrier in preventing water from contacting the wound. The underside of the frame 108 includes adhesive on the side that contacts the skin, and provides some rigidity for the cover to make placement easier. Typically, the dressing permits access to the coupling tips of the catheter through some portion of the dressing, which requires interruption of any adhesive seal formed by the adhesive used to couple the dressing to the patient's skin. As such, this type of dressing provides opportunity for moisture to reach the wound, particularly if the patient attempts to shower or bathe.

With the wound barrier of the invention 100 applied as illustrated in FIG. 1, however, a virtually water tight seal that surrounds the entire site. The adhesive is exposed on the applied side or underside of the wound barrier in the form of a surrounding adhesive border section 104 that sealably adheres to the skin to isolate the inside of the wound barrier 100 from moisture. This seal prevents exposure to water even during, for example, bathing and showering. Non-adhesive portion 102 provides a transparent cover that is highly vapor resistant and yet is also oxygen permeable. The non-adhesive portion 102 is very thin and highly compliant, enabling the wound barrier to accommodate the catheter device 106 and the dressing, as well as to easily conform to the contours of the patient's arm to ensure a strong seal.

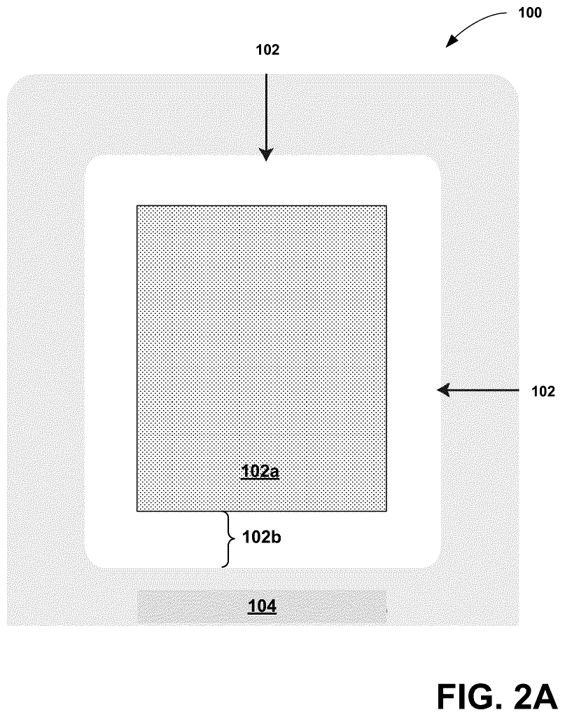

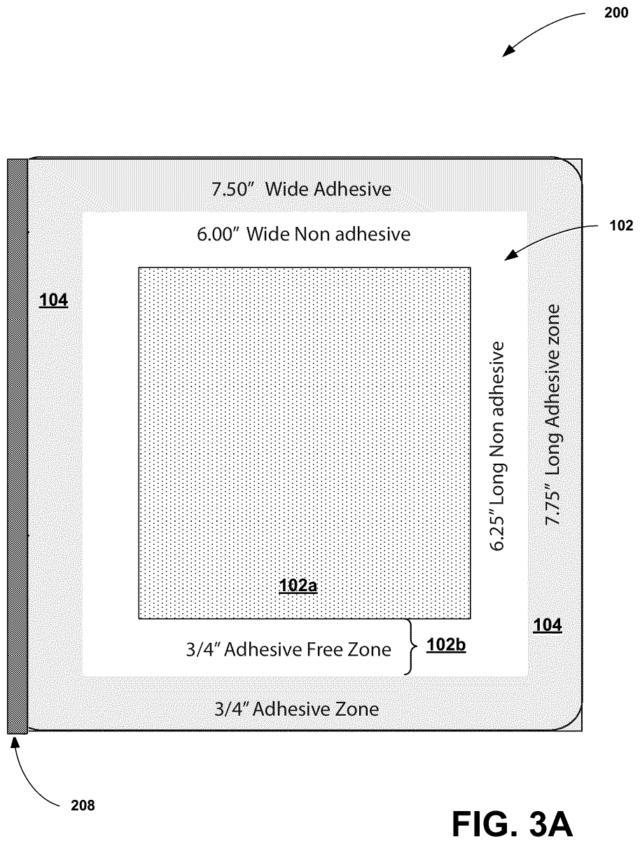

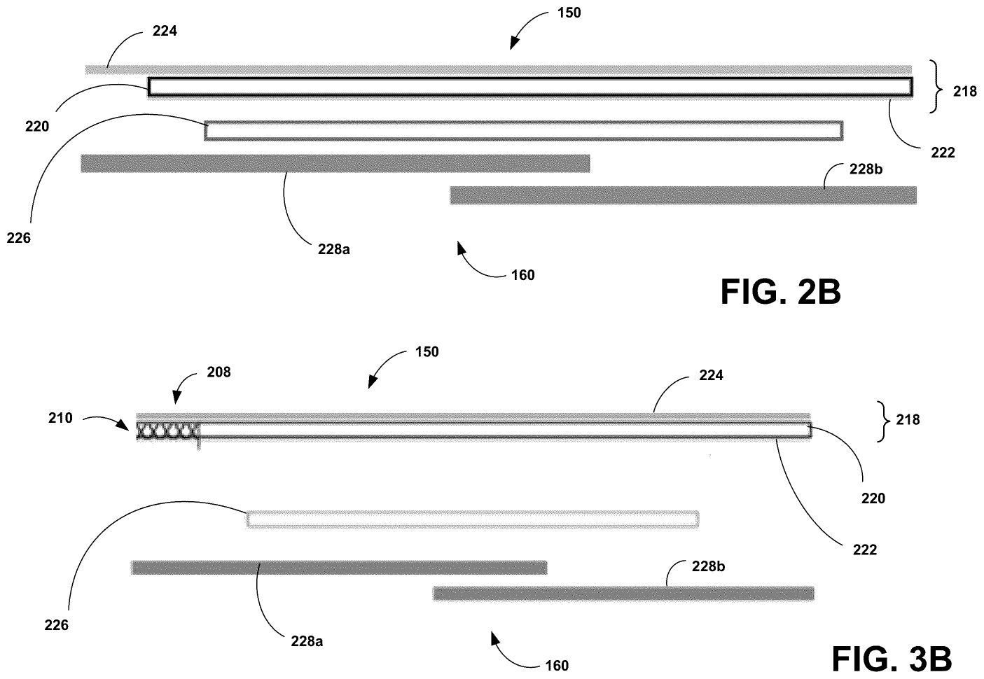

A plan view of an embodiment 100 of the wound barrier of the invention is illustrated in FIG. 2. Wound barrier 100 includes a non-adhesive area or zone 102 that includes a wound site zone 102a having an area that is sufficient to accommodate the area of a wound site of the same or lesser area, and a buffer or clearance zone that extends an additional distance 102b from the wound site and zone 102a that surrounds and extends the non-adhesive area 102. This clearance zone provides an additional buffer to ensure that the non-adhesive zone can clear the volume occupied by devices such as catheters that can be large and variable in size, and might cause unwanted tension due to a large size if additional area for the non-adhesive zone 102 beyond the wound site area 102a is not provided. Wound barrier 100 further includes an adhesive area or zone that surrounds the adhesive free zone and extends substantially to the periphery of the wound barrier 100.

FIG. 2B is a cross-sectional view of the wound barrier of FIG. 2A that illustrates the layers of material that constitute the wound barrier 100 in an exploded view. At the applied surface of the wound barrier 150 (the surface that is applied to the patient's skin) a compound material 218 that can be, for example, a medical tape. One example of such a material is a commercially available single coated polyurethane medical tape marketed by 3M designated as Product Number 9836. Compound material 218 can include a clear poly casting sheet 224 over a thin polyurethane backing layer 220. The polyurethane layer has a layer of pressure sensitive adhesive 222 on the its surface that is opposite from the one with which the casting sheet 224 is in contact. The polyurethane layer 220 while thin and compliant, is not very resistant to moisture.

A clear film 226 is then placed over the adhesive layer 222 of the compound material 218 to “deaden” the adhesive and to form the non-adhesive area 102 and to define the adhesive zone 104 of FIG. 2A. One example of a material suitable for the deadening layer 226 is a polyethylene film commercially marketed by 3M sold under the name Cotran 9720 Backing. The adhesive deadening layer 226 is preferably oxygen permeable, but having a very low MVTR (moisture vapor transmission rate) preferably below 10 grams per meter per 24 hours. The product from 3M has a MVTR of 6. It also has a high oxygen transmission rate of 3840 cc/m/24 hours.

Thus, the combination of the adhesive deadening backing layer 226 and the compound material 218 provides a cover for a wound site that is oxygen permeable and yet highly moisture vapor resistant. Moreover, the adhesive zone 104 that is defined by the combination of the two layers provides a much more reliable adhesive boundary than would a material to which an adhesive zone was patterned onto the surface of material, as voiding at the edges could make the adhesive zone 104 could make the seal much less reliable. Moreover, it permits the use of an existing product that has a uniform layer of adhesive coating on its entire surface. Finally, the combination permits a very thin and compliant material to be used for the compound layer 218, but which does not have very good moisture resistance, by backing it with a backing material that provides a superior MVTR.

Finally, a protective cover or release liner having two overlapping portions 228a and 228b is used to cover the adhesive zone 104 until the wound barrier 100 is ready to be applied. When applying wound barrier 100, one of the overlapping liner sheets is removed first, exposing the adhesive layer 104 for adhesion to the skin. Once the wound barrier 100 is properly placed, the exposed adhesive zone is activated through pressure to the patient's skin. The second backing layer can then be removed so that the remaining adhesive zone 104 is exposed and pressed onto the skin. Finally, the poly casting sheet 224 is then removed from the top surface 150 of the wound barrier, rendering the wound barrier even more compliant.

One advantage of employing removable casting layer 224 is that provides a backing to the thin polyurethane layer 220, which would otherwise be very difficult to handle and properly position when being applied by the healthcare professional.

FIG. 3A illustrates an alternate embodiment 200 of the wound barrier of the invention, where the layers are all the same as previously described, except that a tab 208 is created along one edge of the wound barrier 200 to facilitate the removal of the casting layer 224 after the wound barrier 200 is deployed. The tab 208 is created by scoring away a strip of the polyurethane 220 and adhesive layers 222 under the casting layer 224. The strip can be, for example, about 0.25 inches. FIG. 3A provides exemplary dimensions for one possible embodiment of the wound barrier 200. Those of skill in the art will recognize that the dimensions can be varied in accordance with the size of various wound sites that might be accommodated by the wound barrier of the invention. In addition, while the embodiment shows a rectangular geometry, those of skill in the art will recognize that other geometries may be used without deviating from the scope of the invention.

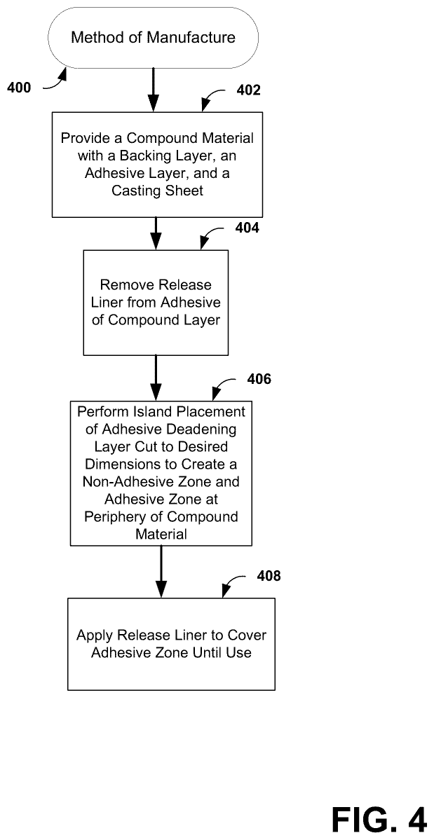

The wound barrier of the invention can be manufactured by the manufacturing process of the invention as illustrated by the procedural flow diagram of FIG. 4. First, a compound material 218 is provided that is suitably compliant and oxygen permeable as previously discussed. If the 3M product is used, the first step would be to remove the release liner (not shown) to reveal the adhesive layer 222. If tab 208 is desired, a strip of the polyurethane and adhesive layers are scored from the compound layer 218 down to the casting layer 224.

An adhesive deadening layer sized to create a desired non-adhesive zone area is then island deposited onto the adhesive layer 222 to define both the non-adhesive zone 102 as well as the adhesive zone 104. The adhesive deadening layer should also be suitably compliant, and have substantial oxygen permeability while exhibiting very low moisture vapor transmission as previously discussed.

Apply 2 webs of, for example 3600 release liner, in an over lapping pattern over the dimensions of the wound barrier to cover the adhesive zone and then die cut the entire wound barrier to the desired dimensions from the compound material.

Those of skill in the art will recognize that Technical Information Sheets are publicly available for the example materials products #9836 (as used for the compound material 218) and #9720 (as used for the adhesive deadening layer 226). Those technical data sheets describe the characteristics of materials suitable for use as those layers, and are hereby incorporated herein by this reference. Those of skill in the art will understand that other commercially available products can be used provided that they meet the characteristics that render them suitable as discussed herein.

1. A wound barrier, configured to attach to the skin around a wound site said wound barrier comprising:
a backing layer comprising an underside and a top surface;
a casting sheet removably attached to the top surface of the backing layer;
an adhesive layer on the underside of the backing layer;
an adhesive deadening layer disposed over a portion of the adhesive layer, such that there is defined a non-adhesive area and an adhesive border that surrounds the non-adhesive area and is substantially at the periphery of the backing layer.

2. The wound barrier, according to claim 1, further comprising a release liner removably attached to the underside of the backing layer and covering the adhesive border.

3. The wound barrier, according to claim 1, wherein the backing layer, casting sheet, and adhesive layer comprise a compound material.

4. The wound barrier, according to claim 3, wherein the compound material is a medical tape.

5. The wound barrier, according to claim 1, wherein the adhesive layer comprises a pressure sensitive adhesive.

6. The wound barrier, according to claim 1, wherein the backing sheet comprises an oxygen permeable polyurethane.

7. The wound barrier, according to claim 6, wherein the adhesive deadening layer is oxygen permeable and has a Moisture Vapor Transmission Rate (MVTR) of below 10 grams per meter per 24 hours.

8. The wound barrier, according to claim 7, wherein the adhesive deadening layer has a Moisture Vapor Transmission Rate (MVTR) of 6 grams per meter per 24 hours and an oxygen transmission rate of 3840 cc/m/24 hours.

9. The wound barrier, according to claim 8, wherein the adhesive deadening layer comprises a polyethylene film.

10. The wound barrier, according to claim 1, wherein the non-adhesive area defined by the adhesive deadening layer comprises a buffer zone that surrounds a wound site zone, such that, when the wound barrier is applied to the skin around a wound site, the buffer zone minimizes the tension applied to the wound site by the wound site zone.

11. A method for covering a wound site, the method comprising:
obtaining a wound barrier comprising,
a backing layer comprising an underside and a top surface;
a casting sheet removably attached to the top surface of the backing layer;
an adhesive layer on the underside of the backing layer;
an adhesive deadening layer disposed over a portion of the adhesive layer, such that the adhesive deadening layer defines a non-adhesive area surrounded by an adhesive border that is substantially at the periphery of the backing layer;
applying the adhesive border to the skin, wherein the non-adhesive area is over the wound site and the adhesive border surrounds the wound site.

12. The method, according to claim 11, wherein the wound barrier further comprises a release liner that covers the underside of the backing layer, including the adhesive border, and the method further comprises removing the release liner prior to applying the adhesive border to the skin.

13. The method, according to claim 12, further comprising removing the casting sheet after the wound barrier is attached to the skin.

14. A method for manufacturing a wound barrier, the method comprising:
obtaining a compound material comprising:
a backing layer comprising an underside and a top surface;
a casting sheet removably attached to the top surface of the backing layer;
an adhesive layer on the underside of the backing layer; and
applying an adhesive deadening layer to the adhesive layer to define a non-adhesive area surrounded by an adhesive border at substantially the periphery of the backing layer.

15. The method, according to claim 14, wherein the compound material further comprises a release liner that covers the adhesive layer and the method further comprises removing the release liner before applying the adhesive deadening layer.

16. The method, according to claim 14, further comprising applying a release liner to the underside of the backing layer to cover the non-adhesive area and the adhesive border.

17. The method, according to claim 14, wherein the compound material comprises 3M™ Single Coated Polyurethane Medical Tape on Poly Carrier with Liner (Product #9836).

18. The method, according to claim 14, wherein the adhesive deadening layer comprises 3M™ CoTran™ 9720 Backing.

19. The method, according to claim 14, further comprising scoring a strip of the adhesive layer to expose a portion of the backing layer to create a tab at an edge of the backing layer.

20. The method, according to claim 14, wherein the wound barrier is 7.5″ wide and approximately 7.75″ long, with a non-adhesive area that is 6″ wide and 6.25″ long.
